(19) United States
(12) Patent Application Publication
(10) Pub. No.: US 2018/0199610 A1
(43) Pub. Date: Jul. 19, 2018

(54) VITAMIN K AND CAPILLARY FUNCTION

(71) Applicant: VITAK B.V., Maastrict (NL)

(72) Inventor: Cornelis Vermeer, Maastricht (NL)

(21) Appl. No.: 15/561,408

(22) PCT Filed: Feb. 22, 2016
(86) PCT No.: PCT/EP2016/053696

§ 371 Date: Sep. 25, 2017

(30) Foreign Application Priority Data
Feb. 20, 2015 (EP) 15156039.8

(51) Int. Cl. A23L 33/15; A61K 31/122; A61K 45/06; A61P 9/14

(52) U.S. Cl. CPC A23L 33/15; A61K 31/122; A61K 45/06; A61P 9/14; A61B 5/02021; A23V 2200/326; A23V 2250/7142; A23V 2250/7144; A23V 2002/00

(57) ABSTRACT

A new indication of vitamin K is disclosed which results in the provision of methods of treatment, both therapeutic and non-therapeutic, as well as functional foods and pharmaceutical and nutraceutical products comprising vitamin K for improving microvascular integrity and capillary structure and function, thus preventing, mitigating, counteracting or curing diseases associated with impaired capillary morphology including the glycocalyx or capillary dysfunction. Preferably, vitamin K is a menaquinone, most preferably selected from the long-chain menaquinones MK-7 through MK-10.

### FIELD OF THE INVENTION

The present invention relates to the fields of nutrition and pharmacotherapy and discloses the beneficial effect of oral vitamin K intake on the structure and integrity of the microvascular and capillary bed, the transport of nutrients, oxygen, waste and CO₂ across the capillary wall, which is essential for a proper function of the organs and tissues in which these capillaries are embedded. Therapeutic and non-therapeutic methods for treatment or prevention as well as pharmaceutical and nutraceutical products comprising vitamin K are disclosed that improve capillary health and function, and prevent, mitigate or ameliorate diseases which are associated with impaired capillary function.

### BACKGROUND ART

The cardiovascular system is a closed loop, in which blood is pumped by the heart into large arteries that branch out and narrow into arterioles, which further branch out and narrow to form a dense capillary network. Whereas the larger vessels have only a transport function for the flowing blood and delivering it into the capillaries (perfusion), the capillary network constitutes the microvascular circulatory compartment which functions as the exchange surface between tissue cells and the bloodstream enabling the delivery of nutrients and oxygen, as well as the removal of waste and carbon dioxide across the capillary wall. Following this exchange, blood passes through post-capillary venules that progressively widen to become large veins, which return blood to the heart. Histologically, capillaries are composed of endothelial cells, pericytes, and layers of basement membrane. The diameter of capillaries ranges from 5 to 20 μm and they connect terminal arterioles and venules. Endothelial cells form the capillary inner lining, whereas pericytes provide support structure for the capillary endothelial cells [Gu, X. et al. Ultrastruct. Pathol. 2012; 36: 48-55; Dore-Duffy, P and Cleary, K. Methods Mol. Biol. 2001; 686: 49-68; Allt, G and Lawrenson, J. G., Cells Tissues Organs 2001; 169-1-11]. In ultrastructural, immunohistochemical and biochemical studies, pericytes show similarity to vascular smooth muscle cells found in arteries, such as containing smooth muscle actin fibers and a large amount of rough plasma reticulum. It also has been demonstrated that under certain conditions MGP is expressed by pericytes [Canfield A. E. et al. FEBS Lett. 2000; 487: 267-271], where it is supposed to inhibit calcification. Since different organs have different needs as they perform specific functions within the body, the endothelial cells lining the microvasculature of these organs have adopted specific molecular, morphological and functional characteristics, a phenomenon called “EC heterogeneity” [Aird, W. C. Circ. Res. 2007; 100: 158-173; Aird, W. C. Circ. Res. 2007; 100: 174-190]. Although the microcirculation makes up the bulk of the circulatory system, its role in the pathogenesis of age-related diseases has been explored far less than that of the macro-circulation. It has been reported that changes in the coronary micro- and macro-circulation parallel those in the retinal microvessels [Nguyen, T. T. and Wong, T. Y. Trends Endocrinol Metab 2006; 17: 262-268], and it has been suggested that microvascular alterations may serve as an early marker of disease processes [Wang, J. J. et al. Am J Epidemiol 2008; 168: 80-88] secondary to the primary pathological process itself, e.g., hypertension, diabetes mellitus or chronic kidney disease. Conversely, pathological changes in the microcirculation might be the primary instigator of disease with subsequent involvement of the macro-circulation such as in the heart, kidney or brain. For instance, some studies demonstrated that retinal arteriolar narrowing is a forerunner of hypertension [Wang, J. J. et al. Am J Epidemiol 2008; 168: 80-88] and that retinal venular narrowing is associated with inflammation, endothelial dysfunction and markers of atherosclerosis [Nguyen, T. T. and Wong T. Y. Trends Endocrinol Metab 2006; 17: 262-268; Wong, T. Y. et al. Lancet 2001, 358: 1134-1140; Wong, T. Y. and Mitchell, P. Lancet 2007, 369: 425-435]. Because the microvasculature is spatially distributed throughout the body and communicates with every tissue, it is of vital importance for proper organ function and human health. Capillaries traverse every tissue and thus transcend all clinical disciplines [Aird, W. C. Circ. Res. 2007; 100: 174-190]; hence capillary health may form a common denominator in quite diverse pathological conditions, such as abnormalities in children observed after extremely premature birth, hypertension, chronic kidney disease, diastolic left ventricular dysfunction, microvascular angina pectoris, obesity, and diabetes mellitus.

There are several techniques to assess capillary function in vivo. One well-known technique is to measure the glomerular filtration rate in the kidneys. For this function the kidneys rely on the afferent and efferent arterioles controlling the filtration at the glomerular capillaries [Chade, A. R. Compr Physiol 2013; 3: 817-831]. The efferent arterioles branch into the peritubular capillary network, which envelops the proximal and distal convoluted tubules and the capillary network adjacent to the loops of Henle and collecting ducts. The rate of creatinine filtration from the blood stream to the urine provides a good estimate for the function of the capillary network in the kidneys, whereas traces of albumin that are set free in the urine (microalbinuria) provide a second measure for the quality of the renal capillary system and provide a scale for grading chronic kidney disease (from grade 1 to grade 5, see also below).

A second, independent, non-invasive measurement to assess the quality of the microvascular bed is the blood flow in or the diameter of the smallest vessels in the retina of the eye. Non-mydriatic retinal photography allows measuring the retinal arterioles and venules, which are indicative for the microcirculation in the brain. Well-known outcome parameters are retinal arteriolar narrowing (RAN) and the arterio-venous ratio (AVR). Using specialized software, retinal imaging can be equipped with a digital camera so that the individual measurements of arterioles and venules can be combined into summary indexes: the central retinal arteriolar equivalent and the central retinal venular equivalent [Liu, Y. P. et al. Artery Res. 2011; 5: 72-77; Sherry, L. M. et al. Clin Exp Ophtalmol. 2002; 30: 179-182].

Still another non-invasive method to assess the quality of the microvascular bed is the recording of capillary density and perfused boundary region (which is inversely correlated with the glycocalyx width) of the sublingual capillaries using a handheld camera interfaced with a laptop running the dedicated software package [Broekhuizen, L. N. et al. Diabetologia 2010; 53: 2646-2655]. By combining these various measures for capillary quality and function, it is possible to estimate the quality of the total microvascular bed and its capacity to exchange nutrients and waste with the surrounding tissues. Impairments in (micro)vascular function and subsequent attenuated increase in postprandial nutrient delivery in the surrounding tissues may be attributed to a lower capillary density and/or impairments in endothelial wall function. An intact glycocalyx is required to allow transcapillary distribution of nutrients into the interstitial fluid. A thicker perfused boundary region (PBR) is associated with endothelial dysfunction in various organs [Groen, B. B. et al. J. Appl. Physiol. 2014; 116: 998-1005].

Finally, plethysmographic acceleration pulse wave meters have been developed based on the teaching of U.S. Pat. No. 4,432,374 (1984), in which the capillary blood flow in the skin is monitored and used to evaluate the condition of the vasculature. Recently, this technology has been miniaturized into finger-tip equipment enabling the microvascular screening to be performed by a simple non-evasive method within five minutes.

Calcification of the large arteries is a hallmark of cardiovascular disease [Raggi, P. et al. Circulation 2000, 101: 850-855; Vliegenthart, R. et al. Stroke 2002, 33: 462-465] and an independent risk factor for myocardial infarction, stroke and cardiovascular death. Vascular smooth muscle cells synthesize a small secretory protein (11 kD), which contains five unusual amino-acids designated as γ-carboxyglutamate (Gla) and which was therefore named Matrix Gla Protein (MGP) [Schurgers, L. J. et al. Thromb. Haemostas. 2008; 100; 593-603; Cranenburg, E. C. M. et al. Thromb. Haemostas. 2007; 98: 120-125]. The synthesis of active MGP requires two posttranslational modifications: γ-glutamate carboxylation in a vitamin K-dependent way and serine phosphorylation. Carboxylated MGP behaves as a potent inhibitor of arterial calcification [Schurgers, L. J. et al. Thromb. Haemostas. 2008; 100; 593-603; Shanahan, C. M. et al. Crit. Rev. Eukaryot. Gene Expr. 1998; 8: 357-375]. In contrast to systemic calcification inhibitors like fetuin-A, MGP is a local inhibitor of tissue calcification, i.e. that it is synthesized at the site where calcification inhibition is needed. Moreover, the presence of precipitated calcium salt deposits in the vasculature results in substantial accumulation of uncarboxylated MGP around the lesion [Cranenburg, E. C. M. et al. Thromb. Haemostas. 2007; 98: 120-125]. Poor vitamin K intake will lead to insufficiency, during which MGP is partly synthesized in a non-carboxylated form that does not inhibit calcification. High circulating levels of uncarboxylated MGP are therefore regarded as an independent risk factor for cardiovascular morbidity and mortality. In this way it can be explained that vitamin K antagonists (oral anticoagulants) induce arterial calcification [Koos, R., et al. Am. J. Cardiol. 2005; 96: 747-749]. PXE and PXE-like syndrome are genetic disorders associated with impaired vitamin K transport (PXE) or vitamin K utilization (PXE-like) and both syndromes were reported to be associated with impaired MGP carboxylation [Boraldi, F. et al. J. Invest. Dermatol. 2013; 133: 946-954; Vanakker, O. M., et al. Am. J. Med. Genet. Part A. 2011; 155: 2855-2859]. The disease is characterized by multiple symptoms including retinopathy, renal dysfunction and skin calcifications. Elastic fiber calcification has been reported as a common denominator. Because in the general population both serine phosphorylation and glutamate carboxylation are incomplete, MGP may occur as desphospho-uncarboxylated MGP (no posttranslational modifications resulting in a completely inactive form), as phospho-carboxylated MGP (posttranslational modification complete resulting in the biologically active form) and many intermediate species. Presently, diagnostic assays have been developed for two circulating MGP fractions, each with a different diagnostic utility: dp-ucMGP and t-ucMGP [Cranenburg, E. C. M. et al. Thromb. Haemostas. 2010; 104: 811-822; EP 1190259 B1; U.S. Pat. No. 674,847 B1]. These assays are briefly described below.

Dp-ucMGP is a marker of actual vascular vitamin K status and is elevated in patients with kidney disease [Schurgers, L. J. et al. Clin. J. Am. Soc. Nephrol. 2010; 5: 568-575; Keyzer, C. A. et al. Am. J. Kidney Dis. 65 (2015) 474-483], heart failure [Ueland, T. et al. Clin. Sci. (Lond) 2011; 3: 119-127], aortic valve stenosis [Ueland, T. et al. J. Intern. Med. 2010; 268: 483-492], vascular disease [Mayer, O. et al. Atherosclerosis 2014; 235: 162-168] or metabolic syndrome [Dam, V. et al. J. Clin. Endocrinol. Metab. Epub. 100 (2015) 2472-2479]; higher circulating levels of dp-ucMGP were associated with the severity of disease or predicted mortality within 2 to 6 years of follow-up both in patients and in the general population [Liu, Y-P. et al. Hypertension 2015; 65: 463-470]. Dp-ucMGP responds quickly to changes in vitamin K intake either by food or food supplements and is generally regarded as an independent risk marker for artery calcification and mortality.

T-ucMGP is a vitamin K-independent marker which is inversely associated with prevalent coronary or aortic calcification (Agatston score) [Cranenburg, E. C. M. et al. Thromb. Haemostas. 2009; 101: 359-366]. Circulating t-ucMGP reflects the large fraction of ucMGP with high affinity for precipitated calcium salts, which explains the inverse association with tissue calcification. It is generally regarded as a disease marker for aortic and peripheral calcification and low circulating levels of t-ucMGP were reported to predict cardiovascular mortality [Parker, B. D. et al. Ann. Int. Med. 2010; 152: 640-648]. In a cohort of 842 cardiovascular patients, t-ucMGP was also reported to be associated with renal function [Parker, B. D. et al. Nephrol. Dial. Transplant. 2009; 24: 2095-2101], but the authors suggested that the underlying mechanism was associated with either (i) genetic abnormalities in their patient cohort, (ii) stiffness of the large arteries leading to renal dysfunction or (iii) absorption of ucMGP onto calcified arteries. A direct association between vitamin K status and the quality or function of the microvascular network was neither taught nor suggested.

In addition to MGP, several other vitamin K-dependent proteins are synthesized in the vessel wall, well-known examples are protein S and Gas6. Protein S serves as a cofactor that enhances the activity of activated protein C (APC) in the proteolytic degradation of blood coagulation factors V and VIII and also has the APC-independent ability to directly inhibit prothrombinase and tenase by direct binding to coagulation factors V, VIII and X [Hepner, M. and Karlaftis, V. Methods Mol. Biol. 2013; 992: 373-381]. Growth arrest-specific gene 6 protein (Gas6) is a ligand for the tyrosine protein kinase receptors Axl, Mer, and Tyro3, and has a regulator function in cell growth and survival [Laurance, S. et al. Adv. Nutr. 2011; 3: 196-203]. Also, Gas6 was recently identified in retinal capillaries [Kim, Y. S., et al. PLoS One. 2014; 9: e83901. doi: 10.1371/journal.pone.0083901]. The importance of the Gla-residues (and thus: the importance of vitamin K) for its cell growth regulatory role is still unclear.

The prior art describes a beneficial effect of vitamin K on the blood flow in the entire vascular system: from the (large) arteries to the arterioles into the capillaries and from there to the venules and veins. Obviously, an obstruction or impairment in this connected system will decrease the total blood flow, also in the capillaries. But the prior art is silent about where this obstruction may take place. From the literature that was available at the priority date, the only conclusion can be that occlusions or impairments in the (large) arteries, such as coronary arteries, renal arteries, carotid arteries, pulmonary arteries, are meant. Obstruction at this level will have major consequences for the blood flow behind the defect, and inevitably will lead to decreased or blocked supply of essential nutrients and oxygen to the target tissues, and to a similar decreased or blocked transport of waste and carbon dioxide. A clear example is myocardial infarction, which is brought about by an occlusion of one of the coronary arteries, resulting in oxygen deprivation of the myocard tissue downstream, leading to necrosis.

WO 2012/161572 A1 (Friesland Brands) discloses food products supplemented with micronutrients comprising vitamin K2, calcium and magnesium in a weight ratio calcium/magnesium <8, which allegedly support, maintain and/or improve blood perfusion. Perfusion is defined herein as the process of the delivery of arterial blood into a capillary bed in mammalian tissues such as brain, kidney, heart, lung, liver, limbs and the gastrointestinal tract.

WO 2013/122465 A1 (Friesland Brands) discloses a functional food composition comprising fat and vitamin K2 in the form of MK-7, wherein the fat comprises at least 36% (w/w) fatty acids, which allegedly supports, maintains and/or improves blood perfusion. Perfusion is defined as in the previous document.

US 2010/0130618 A1 (Vaidya et al.) describes the therapeutic use of vitamin K for improving blood perfusion and ameliorating hypoxia in venous insufficiency.

Venous insufficiency is a disease of the larger veins, often in the legs, and may be caused, e.g., by venous valve leakage, damage or destruction and weakening of the vein walls. This document indicates an effect of vitamin K on the large veins and explains how the impaired blood flow and resulting hypoxia may translate into microvascular damage and poor nutrient supply to the various tissues. However, it is silent about an effect of vitamin K on the microvasculature (arterioles, capillaries and venules) itself.

US 2013/0178522 (Jamison et al.) discloses pharmaceutical compositions comprising vitamin C supplemented with chromium-free vitamin K for treating or preventing NF-κB-mediated disorders.

NF-κB is found in almost all animal cell types and plays a key role in regulating the immune response to infection. Incorrect regulation of NF-κB has been linked to cancer, inflammatory and autoimmune diseases, septic shock, viral infection, and improper immune development. In all conditions ameliorated by the compositions disclosed in US 2013/0178522, vitamin C is an essential component.

US 2005/0074447 A1 (Papas et al.) discloses the use of γ-tocopherol (vitamin E), optionally in combination with α-tocopherol, for preventing macrovascular and microvascular complications in diabetic patients.

US 2010/0247693 A1 (Marini) discloses cosmetic skin care compositions comprising a mixture of azaleic acid, retinol, vitamin K and certain peptides, primarily for improving the appearance of rosacea-affected skin and secondary for treating other skin conditions such as acne, skin discoloration and sun damage.

None of these documents teach or suggest the use or importance of vitamin K for improving microvascular integrity and function, i.e. the transport of nutrients through the capillary wall into the extravascular compartment of the surrounding tissues, and the role of vitamin K outside the vascular system.

U.S. Pat. No. 4,432,374 (Osanai) discloses the technique of monitoring the skin microvasculature using a plethysmographic acceleration pulse wave meter to diagnose malfunction or disease of the circulatory system, including myocardial infarction and cerebral apoplexy. The document is silent, however, about detecting poor vascular vitamin K status or other risk factors for cardiovascular disease and neither teaches or suggests the use of plethysmography for evaluating the risk of vitamin K-insufficiency-associated diseases outside the cardiovascular area.

### SUMMARY OF THE INVENTION

The present invention provides in one aspect a method of treatment for improving the exchange of nutrients, oxygen, carbon dioxide and waste between the blood stream and adjacent tissues across the capillary wall in the microvasculature of a mammalian subject, preferably a human subject, thus optimizing microvascular integrity and capillary structure, morphology, density and function, which comprises administering to said subject an effective amount of vitamin K.

Said vitamin K is selected from phylloquinone (vitamin K1) and menaquinone (vitamin K2), preferably one of the menaquinones (vitamin K2) and combinations thereof, more preferably one of the long-chain menaquinones MK-7, MK-8, MK-9, MK-10, and combinations thereof. In a preferred embodiment of the invention said vitamin K is synthetically prepared.

Said vitamin K is preferably administered in addition to the normal dietary intake of vitamin K as a functional food, a food supplement, a probiotic or a pharmaceutical preparation.

In a preferred embodiment said vitamin K is administered orally. A suitable and preferred dose range is between 5 and 5000 μg/day, preferably between 25 and 1000 μg/day, more preferably between 50 and 500 μg/day and most preferably between 100 and 250 μg/day.

In another embodiment of the invention the method of treatment is provided for mammalian subjects, in particular human subjects suffering from a disease associated with impaired morphology or function of the microvasculature, in particular impaired capillary morphology or capillary dysfunction. The microvasculature may be restricted to the capillary bed.

In still another embodiment of the invention the method of treatment is provided for mammalian subjects, in particular human subjects suffering from a disease selected from renal disease, impaired vision or macula degeneration, left ventricular dysfunction, vascular dementia or cognitive decline, food malabsorption, COPD, pulmonary capillary dysfunction, impaired gas exchange efficiency, infertility or erectile dysfunction, diabetic necrosis and peripheral neuropathy.

The present invention provides in another aspect a pharmaceutical or nutraceutical composition for use in the treatment of improving the exchange of nutrients, oxygen, carbon dioxide and waste between the blood stream and adjacent tissues across the capillary wall in the microvasculature of a mammalian subject, preferably a human subject, suffering from a disease selected from the group of diseases associated with impaired morphology or function of the microvasculature, in particular impaired capillary morphology or capillary dysfunction, renal disease, impaired vision or macula degeneration, left ventricular dysfunction, vascular dementia or cognitive decline, food malabsorption, COPD, pulmonary capillary dysfunction, impaired gas exchange efficiency, infertility or erectile dysfunction, diabetic necrosis and peripheral neuropathy, which comprises as active ingredient at least vitamin K.

In a particular embodiment of the invention said pharmaceutical or nutraceutical composition is used for administration: a) in combination with at least one other food or food supplement known to improve cardiovascular health such as antioxidants, omega-3 fatty acids, niacin, coenzyme Q, inositol, stanols, sterols, or L-arginine; or b) in combination with at least one pharmaceutically or nutraceutically active product such as beta blockers, corticosteroids, ACE inhibitors or cholesterol lowering medication.

In still another aspect of the invention the use of the plethysmographic acceleration pulse wave technique is provided as a screening method for evaluating the risk of vitamin K insufficiency or the risk of developing vitamin K-insufficiency related diseases such as vascular calcification, diabetes, metabolic syndrome, pulmonary disease, chronic kidney disease, macula degeneration, osteoporosis and osteoarthritis.

These and other aspects of the present invention will be more fully outlined in the detailed description which follows.

### BRIEF DESCRIPTION OF THE DRAWINGS

FIG. 1 shows the immunohistochemical detection of different MGP isoforms in the microvasculature in healthy tissue. FIG. 1A: Staining for cMGP in vasa vasorum of the great saphenous vein. Capillaries are lining up and show as small circles in red-brown (arrows). Remarkably most heavy staining was seen in the smallest capillaries. FIG. 1B: Close up of cMGP staining in smallest capillaries. FIG. 1C: Staining for pMGP in vasa vasorum of the great saphenous vein. FIG. 1D: Close up of pMGP staining in smallest capillaries. No staining was observed with antibodies against ucMGP or dpMGP (data not shown).

FIG. 2 shows the immunohistochemical detection of different MGP isoforms in the microvasculature in pathological tissues. FIGS. 2A-2D compare skin biopsies obtained from a healthy subject and from a patient suffering from pseudoxanthoma elasticum, a disease leading to skin calcification, end-stage renal disease and blindness. Data presented in FIGS. 2E-2G were obtained with a skin biopsy from the lower extremities of a type-2 diabetes patient. Data presented in FIGS. 2H-2J were from a chronic kidney patient with calcifications in the kidney tissue. 2A: cMGP staining in capillaries from healthy skin shows high abundance of cMGP. 2B: cMGP staining in capillaries from PXE skin shows absence of cMGP in microvessels from diseased skin. 2C: ucMGP staining in capillaries from healthy skin shows that ucMGP is absent or nearly absent in microvessels from healthy skin. 2D: ucMGP staining of PXE skin shows presence of ucMGP in the diseased state. 2E: Von Kossa staining for calcium precipitates (black). 2F: ucMGP staining showing the abundant presence of ucMGP. 2G: cMGP staining demonstrating that very low levels of cMGP were present. 2H: cMGP staining around a small calcium deposit in the kidney; small arrowhead shows calcium deposit, large arrowhead shows wall of arteriole. 2I: ucMGP staining around the same calcium deposit; small arrowhead shows calcium deposit, large arrowhead shows wall of arteriole. 2J: close-up of ucMGP staining of the microvasculature in the same kidney; arrowheads point to smallest vessels (1-5 μm) in which ucMGP can be identified.

FIG. 3 shows a number of clinical endpoints as a function of dp-ucMGP:eGFR (FIG. 3A), systolic blood pressure (FIG. 3B), diastolic blood pressure (FIG. 3C) and the prevalence of hypertension (FIG. 3D). In all cases data are shown for the entire cohort as well as for the sub-groups with normal and low eGFR.

FIG. 4 shows the association between circulating matrix Gla protein levels and stage of chronic kidney disease. Box plots represent the median, interquartile range, and 5th to 95th percentile interval for dp-ucMGP in all participants (n=1159) and in participants with stages 1 (n=549 [46.8%]), 2 (n=565 [48.1%]), and 3 (n=60 [5.1%]) according to the National Kidney Foundation (KDOQI) guideline (estimated glomerular filtration rate ≥90, 60-89, 30-59 mL/min/1.73 m², respectively). P-values are for the association between stage of chronic kidney disease and circulating dp-ucMGP levels.

### DEFINITIONS AND ABBREVIATIONS

The term “vitamin K”, as used herein, refers to phylloquinone (also known as vitamin K₁); and menaquinone (also known as vitamin K₂). Within the group of vitamin K₂, special reference is made to menaquinone-4 (MK-4) and the long-chain menaquinones (MK-7, MK-8 and MK-9), in particular menaquinone-7 (MK-7). It is generally accepted that the methylated naphthoquinone group, which all K vitamins have in common, is the functional group so that the mechanism of action is similar for all K vitamins. Differences may be expected, however, with respect to intestinal absorption, transport, tissue distribution, and bioavailability.

“Capillaries” are defined as the smallest blood vessels in a mammalian body. These microvessels measure between 5 and 20 μm in diameter and connect arterioles and venules.

The term “microvasculature” is defined as the portion of the circulatory system composed of the smallest vessels, i.e. the capillaries and the connecting arterioles and venules.

The term “perfusion” is defined as the process of delivery of arterial blood into a capillary bed in mammalian tissues such as brain, kidney, heart, lung, liver, limbs and the gastrointestinal tract.

The term “capillary function” is defined as the transport of nutrients and oxygen across the capillary wall into the extravascular compartment of the surrounding tissue, and the transport of waste and carbon dioxide across the capillary wall from the tissues back into the blood stream.

The terms “effective amount” and “therapeutically effective amount” are interchangeable and refer to an amount that results in bringing the desired effect.

The term “vitamin K status” refers to the extent to which various circulating Gla-proteins have been carboxylated. Poor vitamin K status means that the dietary vitamin K intake is insufficient to ensure complete Gla-protein carboxylation. Both ucOC and dp-ucMGP are well recognized as sensitive markers for poor vitamin K status.

In general a distinction is made between hepatic vitamin K status (carboxylation of coagulation factors) and extra-hepatic vitamin K status (carboxylation of Gla-proteins not synthesized in the liver). The liver produces the vitamin K-dependent blood coagulation factors. Insufficient hepatic vitamin K status is extremely rare; therefore, the clotting factors are no sensitive markers for vitamin K status. Unless stated otherwise, the term vitamin K status as used herein refers to both hepatic vitamin K status and extrahepatic vitamin K status.

In the present patent application a person's vitamin K status will be regarded as poor when the circulating levels of uncarboxylated extra-hepatic Gla-proteins MGP (measured as dp-ucMGP) and/or osteocalcin (as measured as ucOC) are above the upper normal level in healthy adults.

The term “Gla” stands for γ-carboxy glutamic acid, an unusual amino acid which is formed posttranslationally by vitamin K action.

RAN stands for retinal arteriolar narrowing, AVR for arterio-venous ratio, and PBR for perfused boundary region.

MGP stands for Matrix Gla-protein, one of the 17 vitamin K-dependent proteins presently known. Mature MGP contains five Gla-residues, four of which are located in what is called the “Gla-domain”, i.e. the amino acid sequence 35-54 in human MGP. Likewise, ucMGP stands for uncarboxylated MGP and cMGP for carboxylated MGP. Mature MGP also contains three serine residues which are phosphorylated. Likewise, pMGP stands for phosphorylated MGP and dpMGP for desphospho MGP (=non-phosphorylated MGP), and dp-ucMGP for desphospho-uncarboxylated MGP. Unless stated otherwise, the term “MGP” as used herein refers to any form of Matrix Gla Protein, either in its carboxylated or uncarboxylated form and either in its phosphorylated or non-phosphorylated form, and mixtures thereof.

GGCX stands for γ-glutamate carboxylase, the vitamin K-dependent enzyme catalysing the posttranslational carboxylation of all Gla-containing proteins.

Warfarin is known as a “vitamin K antagonist” and is a direct inhibitor of the enzyme vitamin K-epoxide reductase (VKOR), which is responsible for the recycling of vitamin K into the active form vitamin K hydroquinone. Once VKOR is inhibited, vitamin K can only be used once, resulting in a 1000-2000 fold increased vitamin K requirement.

For the purposes of this specification and the appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters set forth, the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a monomer” includes two or more monomers.

### DETAILED DESCRIPTION OF THE INVENTION

The main function of arteries and veins is the transport of blood, whereas the most important function of capillaries is to form the exchange surface for delivery of nutrients and waste removal between tissue cells and the bloodstream. Whereas for the large arteries dp-ucMGP and t-ucMGP have proved to be effective markers for arterial health and quality, the key of our present invention is that vitamin K status as deduced from circulating dp-ucMGP is also (or even: mainly) an effective marker for the (dys)function of the capillaries and for related diseases, i.e. diseases of the organs embedding these microvessels resulting from poor nutrient exchange with the blood stream. Since dp-ucMGP is a risk marker for disease progression and mortality, our findings imply that increased vitamin K intake is a rational strategy for reducing disease progression in a wide variety of organs/tissues, thus improving quality of life and increasing life expectancy. The key findings leading to this conclusion are summarized below.

### I. At Tissue Level Vitamin K Status is Important for Microvascular Health.

In healthy tissues, different MGP conformations were identified in capillaries by immunohistochemical techniques using conformation-specific antibodies. Most prominent conformations were carboxylated MGP (cMGP) and phosphorylated MGP (pMGP), suggesting that mature MGP is the predominant form in the healthy microvasculature. Tissues examined include kidney, skin, retina and vasa vasorum of the great saphenous vein. Surprisingly, we found uncarboxylated MGP (ucMGP) to be a major or even the predominant conformation in capillaries obtained from various pathologies including renal disease and diabetes: ucMGP was frequently associated with (micro)calcifications present in or around the capillaries, which is suggestive of a local severe vitamin K insufficiency in association with various diseases. Collectively, these data demonstrate that MGP carboxylation is a measure for the quality of the microvascular and capillary network. These data do not show a causal relationship between MGP carboxylation and the microvasculature, however. Alternatively, dp-ucMGP may be a surrogate marker for carboxylation of other vitamin K-dependent proteins such as protein S or Gas6.

II. In the Circulation, Dp-ucMGP (a Marker for Vascular Vitamin K Status) is Associated with the Progressive Decrease of Capillary Structure and Function.

Whereas in the published literature dp-ucMGP was mainly found to be associated with poor arterial health and mortality, we have surprisingly found that in the healthy population it also predicts loss of organ function, such as a decrease of the estimated glomerular filtration rate (eGFR), left ventricular diastolic dysfunction and impaired retinal blood flow. In a large-scale family-based cohort known as the FLEMENGHO cohort [the cohort is described by Liu et al. Hypertension 65 (2015) 463-470] we have investigated the association between circulating dp-ucMGP (i.e.: vitamin K status) and (i) microvascular morphology using non-mydriatic retinal photography and sublingual video-microscopy recording; (ii) microvascular function by assessing the eGFR and renal resistive index as measures for kidney function, and the left ventricular diastolic function as a measure for cardiac function. The data showed a marked association between vitamin K status and capillary diameter, density and integrity, including the estimated glycocalyx width and both renal and left ventricular function. This demonstrates that besides the quality of arteries, also the quality and function of the capillary bed are dependent on vitamin K.

Taken together, these data demonstrate that vitamin K is required for microvascular health and function, and that poor vitamin K status is a an independent risk factor for microvascular pathologies including microcalcification in or around the capillary bed, arteriolar narrowing, capillary rarefaction and impaired glycocalyx, leading to compromised delivery of nutrients and waste removal between tissue cells and the blood stream. Tissues that may especially be affected by impaired capillary function are those with high capillary density, including: kidney, brain, eye, myocard, liver, lung, intestine, skin, pancreas and testis, leading to diseases such as: chronic kidney disease, cognitive decline/vascular dementia, impaired vision/macula degeneration, heart failure, left ventricular dysfunction, food malabsorption, pulmonary capillary dysfunction, impaired gas exchange efficiency, male infertility or erectile dysfunction, diabetic necrosis, diabetic peripheral neuropathy and many others.

The strong association between a) poor vitamin K status and b) impaired microvascular structure, integrity and function also implies that impairments diagnosed in the microvascular structure (as may be found, for instance, by non-mydriatic retinal photography, sublingual capillary density recording or plethysmographic acceleration pulse wave monitoring in the microvascular blood flow in the skin) may be an indication for vitamin K insufficiency and vitamin K insufficiency-related diseases, including vascular calcification, diabetes, metabolic syndrome, pulmonary disease, chronic kidney disease, macula degeneration, osteoporosis and osteoarthritis.

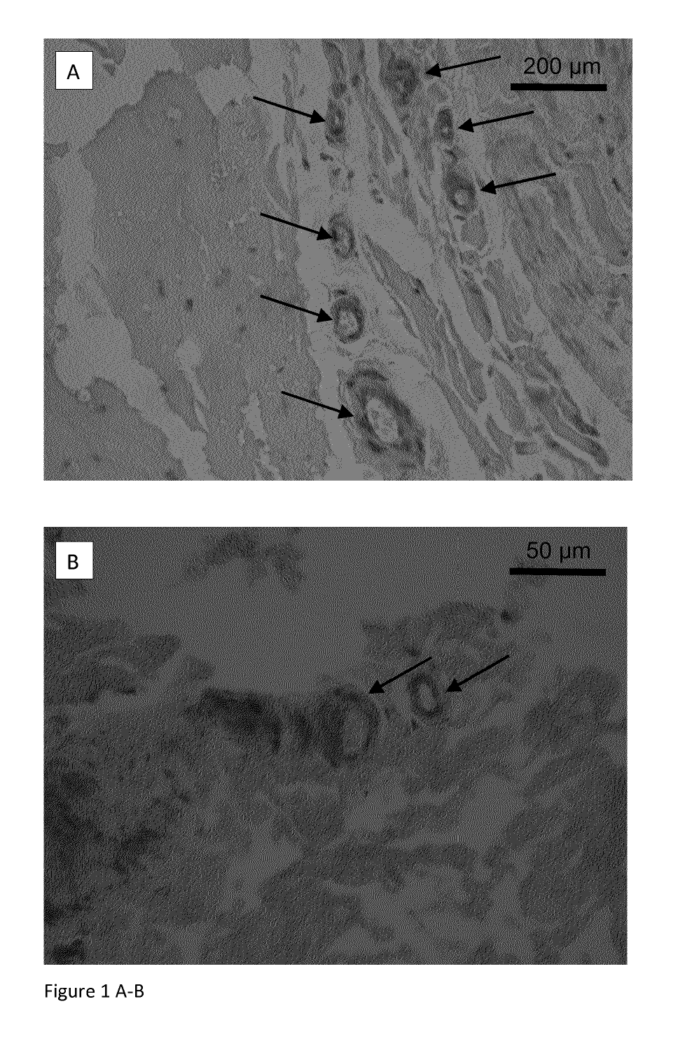

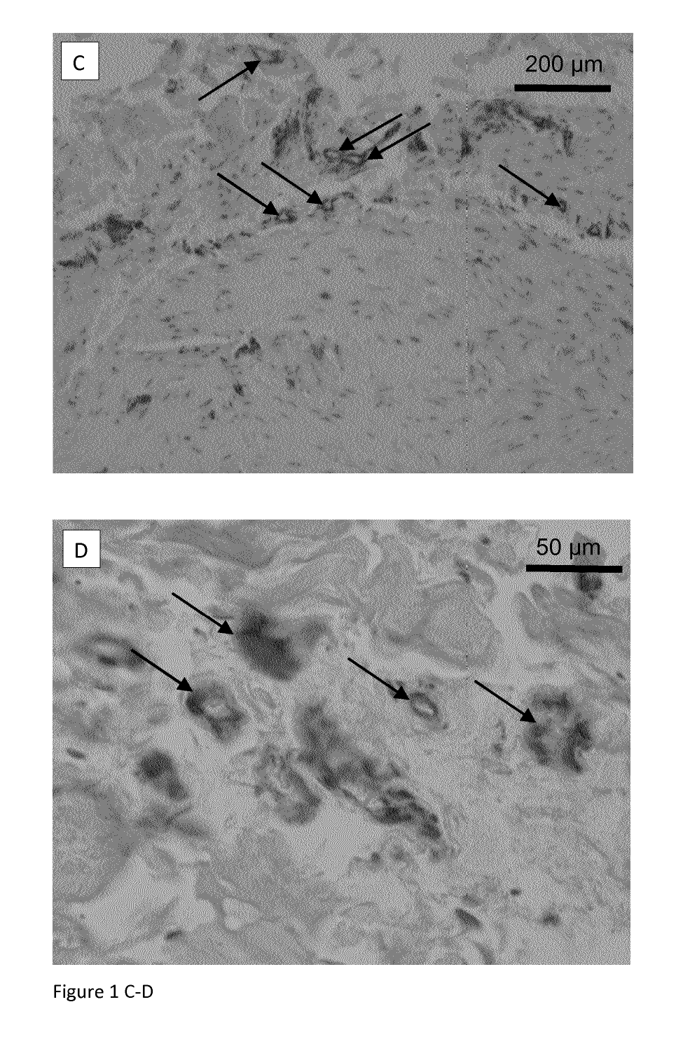

The present invention is based on the identification of both cMGP and pMGP in the wall of the healthy microvasculature and capillaries (see FIG. 1). These are the smallest blood vessels in the human body and have a unique function: the exchange of nutrients and waste with the surrounding tissues. Larger vessels, i.e. arteries and veins, only have a transport function for the blood flow. Whereas it has been well described that the vascular smooth muscle cells in the arteries produce MGP which acts as a local calcification inhibitor, notably in the tunica media, it was surprisingly found that also capillaries—which do not contain smooth muscle cells—produce MGP. Since the capillary bed comprises more than 95% of the total vascular surface, it is evident that by far most of the circulating MGP originates from the microvasculature, and not from the larger vessels.

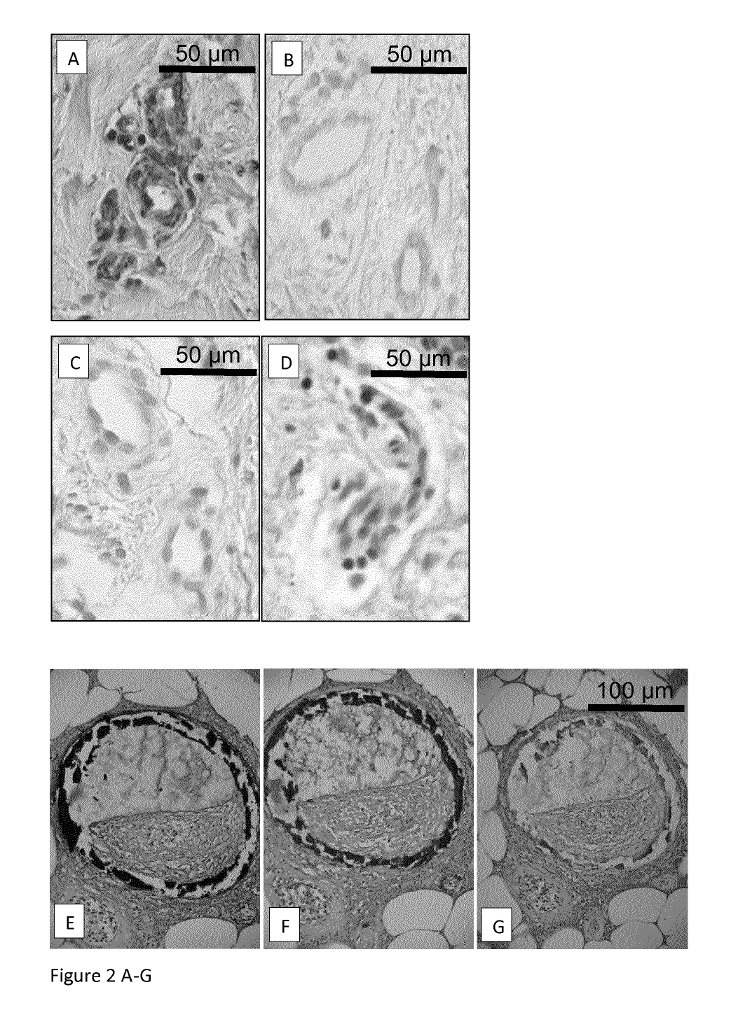

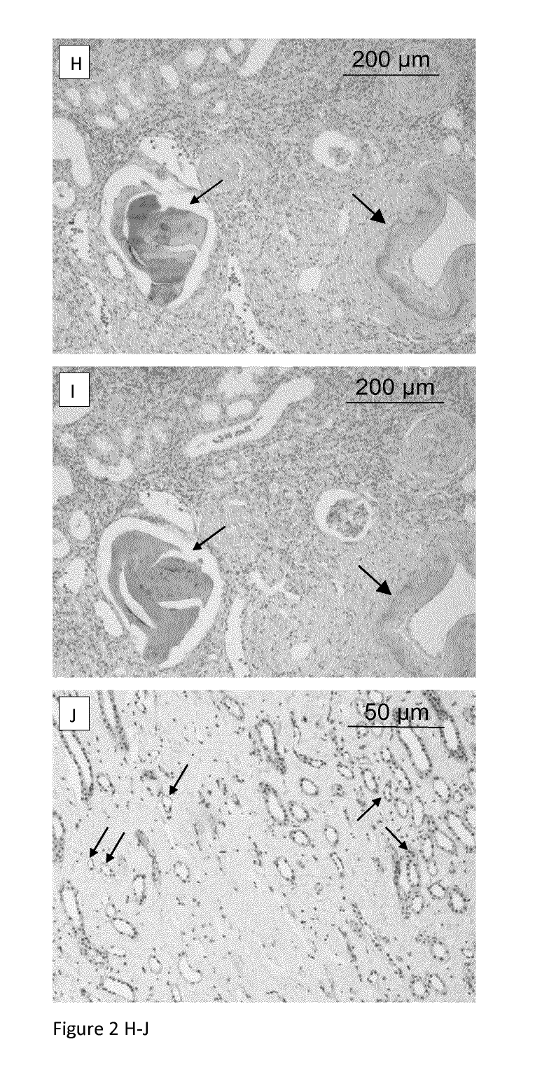

The present invention is further based on the surprising identification of ucMGP in the microvasculature and capillaries of tissues obtained from a number of patients. Unexpectedly, in some cases ucMGP was even the predominant isoform in the capillary wall, and even further accumulated at sites of microcalcification. This was demonstrated in skin biopsies obtained from a patient suffering from pseudoxanthoma elasticum (see FIGS. 2A-D), skin biopsies from a type-2 diabetic patient (FIGS. 2E-G), and kidney biopsies from patients with end stage renal disease (FIGS. 2H-J). In the latter case it is noteworthy that the calcification is associated with vitamin K-insufficiency outside the vascular bed, so within the tissue. This is consistent with impaired transport of vitamin K over the capillary wall.

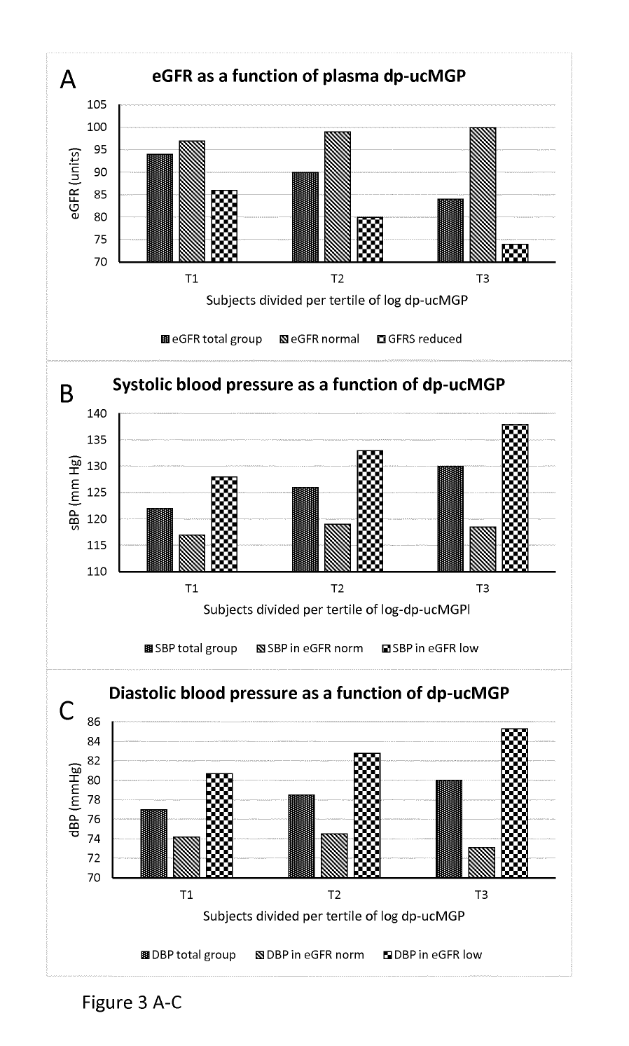

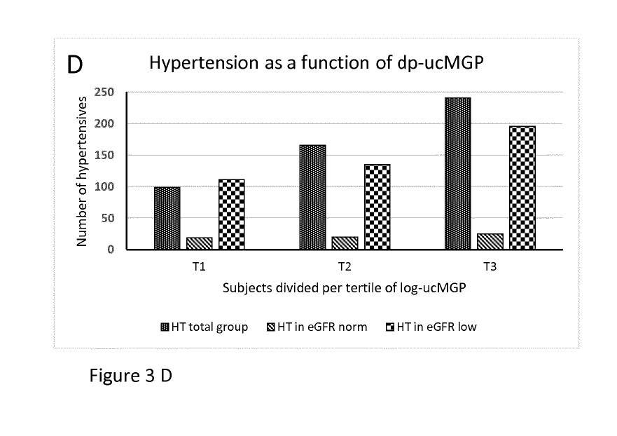

We have surprisingly found that in the healthy population vitamin K status, as measured by circulating dp-ucMGP, is inversely associated with the capillary function, as determined from the estimated glomerular filtration rate (eGFR, see FIG. 3A). This was tested in the previously described FLEMENGHO cohort [Stolarz-Skrzypek, K. et al. J. Am Med. Ass. 2011; 305: 1777-1785], which is a large-scale family-based study for which the recruitment started in 1985. Blood pressure is strongly associated with capillary quality and integrity and consistently we have also found that vitamin K status is strongly and inversely associated with blood pressure and with the incidence of hypertension (see FIGS. 3B, 3C and 3D).

We have also surprisingly found that vitamin K status is inversely associated with the quality of the retinal vasculature, as measured from the retinal arteriolar narrowing (RAN) by using non-mydriatic retinal photography, which was performed in a subgroup of the total FLEMENGHO cohort (n=145). Increased circulating dp-ucMGP levels were associated with lower average arterio-venous ratio (AVR): 0.87, 0.83 and 0.75 for the lowest, middle and highest tertile of dp-ucMGP respectively; and with more microvessels with an AVR<0.8, which is the generally accepted lower limit for healthy capillaries (15.1%, 17.0% and 23.7% for the lowest, middle and highest tertile of dp-ucMGP respectively). This demonstrates that vitamin K is important for the blood supply of the retina and consequently with its health and function.

In addition, vitamin K status was found to be inversely associated with the quality of the sublingual capillary network, as was determined in a subgroup of the FLEMENGHO cohort (n=83). We found that higher circulating levels of dp-ucMGP are associated with a higher capillary recruitment as exemplified by a higher erythrocyte-perfused capillary density (316-335-349/mm² for the lowest, middle and highest tertile of plasma dp-ucMGP respectively). Moreover, the perfused boundary region (PBR) increased with increasing dp-ucMGP, reflecting impaired endothelial glycocalyx width and a compromised nutrient exchange over the endothelium.

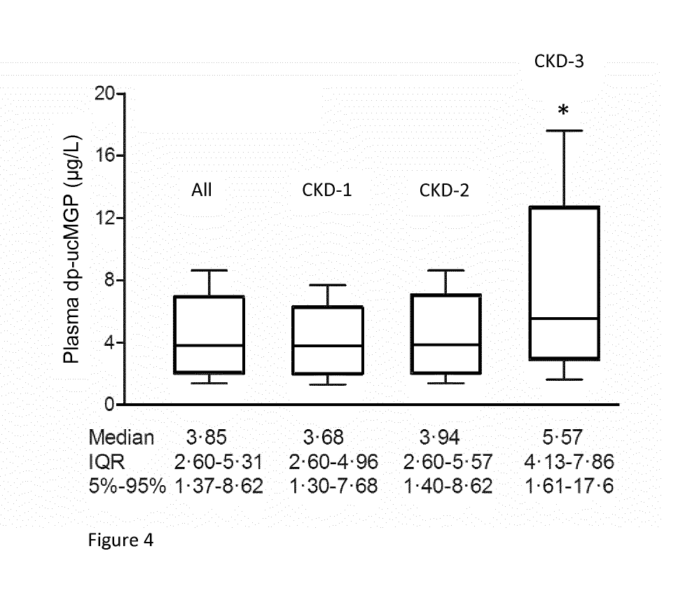

In the healthy population, dp-ucMGP appears to be associated with phases 1, 2 and 3 of end-stage renal disease classified according to the National Kidney Foundation (KDOQI) guideline. Estimated glomerular filtration rate, ≥90, 60-89, and 30-59 mL/min/1.73 m², respectively (see FIG. 4).

In accordance with the present invention vitamin K is used to improve capillary health and function, i.e. the exchange of nutrients and waste with the tissues surrounding the microvasculature. The implication of this finding is a completely new field of application of vitamin K, namely its use for improving the health and function of organs and tissues, notably tissues with a high microvascular density, including kidney, lung, brain, myocard, retina, testes, pancreas, liver and skin. Oral applications of K vitamins in the form of pharmaceutical or nutraceutical preparations, food supplements or functional foods are preferred.

For use in the present invention, phylloquinone (vitamin K1), MK-4 and the long-chain menaquinones MK-7, MK-8, MK-9 and MK-10 are preferred, of which MK-7 is particularly preferred.

In preparations for oral use the dose of vitamin K effective in performing the invention is not restricted but may be established individually under guidance of the extent to which circulating dp-ucMGP, ucOC or other uncarboxylated Gla-proteins are decreased as a result of the treatment. Current AI values or Adequate Intakes (as determined by the Institute of Medicine) are 120 μg for healthy men and 90 μg for healthy women. Where national legislation permits, it may be advisable to provide dosage ranges between 5 μg/day and 5 mg/day, preferably between 25 μg/day and 1000 μg/day, more preferably between 50 μg/day and 500 μg/day, and most preferably between 100 and 250 μg/day. For patients with a known poor vitamin K status, including those with gastrointestinal disease, food malabsorption, bile obstruction, chronic kidney disease, diabetes mellitus, coronary artery calcification, calciphylaxis, or peripheral artery disease, these ranges are preferably twice as high.

The duration of the intervention is preferably lifelong. In terms of body weight, daily dosages may range between 0.05 and 50 μg/kg body weight, preferably between 0.25 and 10 μg/kg body weight, more preferably between 0.5 and 5 μg/kg body weight and most preferably between 1 and 2.5 μg/kg body weight.

Vitamin D may be included together with vitamin K in the compositions described in this invention since it is well known that both osteocalcin and MGP have a vitamin D-responsive element in their promoter sequence and that expression of these proteins may thus be stimulated by vitamin D. Any form of natural or synthetic vitamin D may be employed, including vitamin D₁, vitamin D₂ (calciferol), vitamin D₃ (cholecalciferol) and vitamin D analogues (e.g. alfacalcidol, dihydrotachysterol, calcitriol). Natural sources of vitamin D, include saltwater fish, organ meats, fish-liver oils and egg yolk. Suitable dosages of vitamin D are 2 to 50 μg/day, preferably 5 to 20 μg/day, and most preferably about 7 to 10 μg/day.

Vitamin K-enriched nutritional products may be manufactured to provide the daily requirements of vitamin K. For example, vitamin K may be added to food products, such as, for example, meal replacers, ice cream, sauces, dressings, spreads, bars, sweets, snacks, cereals, beverages, etc., by methods as described in EP 1153548 and U.S. Pat. No. 8,354,129, the entire disclosure of which is incorporated by reference herein. Vitamin K may also be used in food supplements such as multivitamins, tablets, capsules, sachets, and other forms.

Sources of vitamin K which can be used according to the present invention include the following: phylloquinone from natural sources, such as vegetable extracts, fats and oils, synthetic phylloquinone, different forms of vitamin K₂: synthetic MK-4, MK-5, MK-6, MK-7, MK-8, MK-9, MK-10, MK-11, MK-12 and MK-13, natto (food prepared from fermented soy-bean, rich in MK-7), natto extracts, and other fermented foods or dairy products, menaquinones produced by deep tank fermentation followed by various extraction steps, and menaquinones derived from the application of pre- and probiotics.

Vitamin K is conventionally provided in the form of tablets or capsules, i.e. in a pharmaceutical or dietary supplement format. For pharmaceutical preparations or dietary supplements the vitamin K may be compounded with pharmaceutically acceptable carriers, excipients or diluents in the forms of pills, tablets (coated or uncoated), hard or soft capsules, dragées, lozenges, oral solutions, suspensions and dispersions, syrups or sterile parenteral preparations. Suitable excipients include inert diluents, such as calcium carbonate, sodium carbonate, lactose, calcium phosphate, sodium phosphate; granulating and disintegrating agents, such as cornstarch or alginic acid; binding agents, such as starch gelatine or acacia; effervescents; and lubricating agents, such as magnesium stearate, stearic acid or talc. Since vitamin K is a fat-soluble vitamin, it is preferably formulated in oil, for instance fish oil or sunflower seed oil and manufactured in the form of soft or hard gelatin capsules. If protected from light, all forms of vitamin K are stable at room temperature with no significant loss after 2 years of storage. These products can be prepared by conventional manners known in the art and typically have a weight between 100 milligrams and 1 gram. Moreover, it is well known that through nano-encapsulation fat-soluble vitamins including vitamin K can be prepared in a water-soluble form. The effective dose of vitamin K for oral use per capsule or tablet is typically between 5 and 5000 micrograms, preferably between 25 and 1000 micrograms, more preferably between 50 and 500 micrograms and most preferably between 100 and 250 micrograms.

Vitamin K may also be provided via probiotic preparations comprising (or promoting the growth of) menaquinone-producing bacteria, which include Lactococcus lactis lactis, Lactococcus lactis cremoris, Leuconostoc lactis, Leuconostoc mesenteroides, Brevibacterium linens, Brochontrix thermosphacta, Hafnia alvei, Staphylococcus xylosus, Staphylococcus equorum, Arthrobacter nicotinae, Bacillus subtilis natto, Propioni shermanii, Propioni reichmanii, Arachnia propionica, Veillonella parvula, Bacteroides fragilis, Bacteroides disens, Bacteroides bivius, and Klebsiella pneumonia.

It is also possible to deliver or administer vitamin K, optionally together with vitamin D, in a fortified food or beverage product. Preferred nutritional product formats include: juice drinks, dairy drinks, powdered drinks, sports drinks, mineral water, soy beverages, hot chocolate, malt drinks, biscuits, bread, crackers, confectioneries, chocolate, chewing-gum, margarines, spreads, yoghurts, breakfast cereals, snack bars, meal replacements, protein powders, desserts, and medical nutrition-tube feeds and nutritional supplements.

Vitamin K may also be delivered or administered, optionally together with vitamin D, in combination with other health foods or supplements, for example omega-3 fatty acids, anti-oxidants (vitamin C, vitamin E), niacin, coenzyme Q, inositol, stanols, sterols and L-arginine.

It is also possible to deliver or administer vitamin K, optionally together with vitamin D, in combination with medicines which are generally used to treat or cure microvascular disease or to improve capillary function such as—but not limited to—anti-inflammatory drugs (e.g. corticosteroids), anti-ischemic drugs (e.g. ranolazine), vasodilative drugs (e.g. ACE inhibitors, quinapril), angiotensin II receptor blockers (azilsartan, candesartan, eprosartan, irbesartan and similar compounds), and statins.

Moreover, K vitamins may also be used in combination with medicines which are generally used to maintain, promote or cure the function of a wide variety of tissues, for example kidney, brain, eye (retina), myocard, liver, lung, intestine, skin, pancreas and testis.

The invention further relates to the use of the plethysmographic acceleration pulse wave technique as a screening method for evaluating the risk of vitamin K insufficiency or the risk of developing vitamin K-insufficiency related diseases such as vascular calcification, diabetes, metabolic syndrome, pulmonary disease, chronic kidney disease, macula degeneration, osteoporosis and osteoarthritis, and relates also to an apparatus for screening the blood flow acceleration and blood vessel flexibility using the Acceleration Plethysmography (APG) technique.

APG has been widely known as a conventional non-invasive and simple method to obtain information on peripheral circulatory kinetics. Light radiating from the light source penetrates the fingertip, and a light interceptor on the opposite side detects the volume of the transmitted light. The radiated light is absorbed by hemoglobin carried in the bloodstream, and the remainder enters the light interceptor as transmitted light. We have demonstrated a strong association between APG and dp-ucMGP, i.e. vitamin K insufficiency. Therefore, the APG technique may be used as a point-of-care technique for a first screening of subjects suspected of vitamin K insufficiency.

Having now generally described the invention, the same may be more readily understood through the following reference to the experimental part including the examples, which are provided by way of illustration and are not intended to limit the present invention unless specified.

### EXPERIMENTAL

### Materials and Methods

Immunohistochemistry:

Tissue biopsies were embedded in paraffin according to standard procedures and sections were prepared for immunohistochemistry. Staining was performed with monoclonal antibodies against uncarboxylated MGP (ucMGP), carboxylated MGP (cMGP), desphospho-MGP (dpMGP) and phosphorylated MGP (pMGP). These antibodies were used in dilutions ranging between 1 and 10 μg/mL (as empirically determined to be optimal) in phosphate-buffered saline according to standard techniques.

Population-Based Studies:

This research was performed in the previously described FLEMENGHO cohort [Stolarz-Skrzypek, K. et al. J. Am Med. Ass. 2011; 305: 1777-1785], which is a large-scale family-based study for which the recruitment started in 1985. The Ethics Committee of the University of Leuven approved the protocol. All participants gave informed written consent. The participation rate at enrolment was 78.0%. Of 3343 participants, 2329 had a plasma sample available in the FLEMENGHO bio-bank, of whom 1179 had plasma MGP, serum & urinary creatinine concentration and 24-h microalbuminuria measured at the same occasion. Five participants were excluded from analysis because they were taking warfarin (n=2), which substantially elevates the concentration on inactive MGP, or because their MGP levels were more than 3 SDs away from the population mean (n=3). Thus, the number of participants analyzed totaled 1174.

Clinical Measurements:

Before the participants were examined at the field center, they were asked to refrain from heavy exercise, smoking, drinking alcohol or caffeine-containing beverages for at least 3 hours. Their blood pressure was measured five times consecutively after they had rested for 5 minutes in sitting position. Each participant's blood pressure was the average of five consecutive auscultatory readings obtained with a standard sphygmomanometer. Hypertension was a blood pressure of at least 140 mm Hg systolic or 90 mm Hg diastolic, or use of antihypertensive drugs. Trained nurses also took a standardized questionnaire inquiring into each participant's medical history, smoking and drinking habits, and intake of medications. Body height and waist circumference were measured to the nearest 0.5 cm. Body mass index was weight in kilograms divided by the height in meters squared.

Fundus photography of the central retina was recorded using a non-mydriatic camera, and all images were evaluated by one experienced reader. Retinal arteriolar narrowing (RAN) is a well-recognized marker of microvascular damage and was established from the arterio-venous ratio (AVR). An AVR<0.8 was defined as decreased. Data were adjusted for age, sex, cigarette smoking, alcohol consumption, and intake of beta-blockers. The sublingual microvascular system was visualized using sidestream dark-field (SDF) imaging. This technique allows imaging of perfused blood vessels covering the entire range of capillary diameters (5-20 μm). SDF measurements were performed in the sublingual oral cavity, an area that is considered as a valid derivative to assess microvascular function in other tissues. Microcirculatory parameters were measured using a handheld Video Capillary Microscope (KK Research Technology Ltd., Honiton Town, Devon, United Kingdom) in combination with dedicated software (Glycocheck ICU, Glycocheck B.V., Maastricht, The Netherlands) to determine a) the number of capillaries per diameter class per square millimeter and b) the endothelial glycocalyx width, which is inversely proportional to the perfused boundary region (PBR). Vessel segments were classified according to their width, (5-20 μm with 1 μm increments. Median and average PBR values were determined for each diameter subgroup over the entire 5- to 20-μm diameter range.

Biochemical Measurements:

Plasma glucose, total and high-density lipoprotein (HDL) serum cholesterol, and serum creatinine were measured using automated methods in a single certified laboratory. Diabetes mellitus was a fasting or random glucose level exceeding 126 or 200 mg/dL (7.0 or 11.1 mmol/L), or use of antidiabetic agents [Expert Committee on the Diagnosis and Classification of Diabetes Mellitus. Diabet. Care 2003; 26: S5-S20]. Participants collected a timed 24-h urine sample for the measurement of microalbumin and creatinine. eGFR was calculated from serum creatinine, according to the Chronic Kidney Disease Epidemiology Collaboration (CKD-EPI) equation [Levey, A. S. et al. Ann. Intern. Med 2009; 150: 604-612]. Chronic kidney disease (CKD) was staged according to the National Kidney Foundation (KDOQI) guideline [Levey, A. S. et al. Kidney Intern 2005; 67: 2089-2100], as eGFR ≥90, 60-89, 30-59 mL/min/1.73 m². Dp-ucMGP was quantified in citrate plasma samples by a pre-commercial ELISA kit [Cranenburg, E. C. M. et al. Thromb. Haemostas. 2010; 104: 811-822].

Statistical Analysis:

For database management and statistical analysis, we used the SAS system, version 9.3 (SAS Institute Inc., Cary, N.C.). Significance was a two-tailed α-level of 0.05 or less. Means and proportions were compared using the large-sample z-test or ANOVA and Fisher's exact test, respectively. The normality of distributions was assessed by the Kolmogorov-Smirnov's test and logarithmically transformed non-normal distributions.

### Introductory Observations

In a first set of experiments we have performed immunohistochemical staining on sections from various human tissues using the four conformation-specific antibodies against MGP species: dpMGP, pMGP, ucMGP and cMGP.

It was found that in microvessels and capillaries from healthy tissues, substantial amounts of MGP were present and that the predominating forms were pMGP and cMGP, thus demonstrating that mature MGP (both carboxylated and phosphorylated) is the common form in the healthy capillary wall. It was also found that the staining in the smallest vessels was more intense than in the walls of larger vessels (both arteries and veins). These results are shown for the capillaries in the vasa vasorum of healthy veins (FIG. 1) and were confirmed in sections from kidney, skin and lung. The high local MGP expression demonstrates its importance for the structure and/or function of the microvasculature, including capillaries with a diameter between 5 and 20 μm.

Similar experiments were performed on sections from pathological tissues. Microcalcifications were found in and around the microvasculature; also here high amounts of MGP were identified, but in the affected vessels the predominant form was ucMGP, with relatively little cMGP present. The highest concentrations of ucMGP were found in association with calcium deposits. Calcium deposits were also found in the tissue at the outside of the vascular bed, notably in association with poor capillary vitamin K status. Such calcium deposits were invariably strongly stained for both cMGP and ucMGP, demonstrating (i) upregulation of MGP synthesis in the cells surrounding the calcification and (ii) insufficient vitamin K supply to ensure full MGP carboxylation (=activation). Tissues examined were kidney (ESRD), skin (diabetes mellitus and PXE) and myocard (heart failure). An example is given in FIG. 2. These data demonstrate that local vitamin K deficiency may result in poor organ function and disease.

In the FLEMENGHO cohort we have further found a strong inverse association between dp-ucMGP and kidney function (eGFR), a positive association between dp-ucMGP and systolic & diastolic blood pressure as well as the risk of hypertension, and an inverse association with measures for retinal and sublingual capillary health. Finally, dp-ucMGP was also associated with the level of chronic kidney disease in preclinical phases of the disease (stage 1, 2 and 3).

In a series of subsequent experiments, examples of which are detailed below, we demonstrate that vitamin K status, as measured by dp-ucMGP, is a strong determinant for the morphology, the function and the density of capillaries, and that vitamin K-insufficiency may lead to compromised capillary function, i.e., nutrient exchange with the surrounding tissues.

### Example 1

The occurrence of different isoforms of MGP was demonstrated in the microvasculature (including the capillaries) of healthy tissues. For the experiment depicted in FIG. 1, we obtained sections from the great saphenous vein of healthy donors. Tissues were fixed in formaldehyde and embedded in paraffin according to general procedures and sections were incubated with conformation-specific monoclonal antibodies against either dpMGP, pMGP, ucMGP or cMGP. Staining was performed with biotinylated rabbit-antimouse IgG and streptavidin-HRP. Whereas the large venous wall was negative for all MGP conformations, pMGP and cMGP were identified in the capillaries of the vasa vasorum, demonstrating that only mature MGP is present in these capillaries. No dpMGP or ucMGP were present in healthy capillaries. Remarkably heavy staining was seen in the smallest capillaries.

### Example 2

The occurrence of different isoforms of MGP was demonstrated in the microvasculature of pathological tissues. For the experiments depicted in FIG. 2, we obtained skin sections from healthy donors, from a patient suffering from pseudoxanthoma elasticum, from the lower extremities of a patient with severe type-2 diabetes mellitus, and kidney sections from a chronic kidney disease patient. The tissues were fixed in formaldehyde and embedded in paraffin according to general procedures and sections were incubated with conformation-specific monoclonal antibodies against either dpMGP, pMGP, ucMGP or cMGP. Staining was performed with biotinylated rabbit-antimouse IgG and streptavidin-HRP. Capillaries were readily visible in the skin tissue. In the healthy skin, cMGP was the most predominant form, whereas in PXE it was ucMGP. Remarkably, in some cases ucMGP was the most predominant conformation, notably in association with calcification, demonstrating a local vitamin K deficiency in in these capillaries. FIG. 2 H-J finally demonstrates that poor capillary vitamin K status is associated with calcification outside the vascular bed, in the renal tissue.

### Example 3

Dp-ucMGP was measured in all eligible participants in the FLEMENGHO cohort and the associations with clinical outcomes were evaluated.

FIG. 3A: the estimated glomerular filtration rate (eGFR) was measured in the total group, and subdivided in those with a normal eGFR and those with decreased eGFR. It was found that eGFR was lower at increasing tertiles of dp-ucMGP (=decreasing vitamin K status); this was found in the total group and even more prominent in the group with low eGFR (p<0.001 in both cases) but not in the group with normal eGFR. This demonstrates that poor capillary vitamin K status affects eGFR.

FIG. 3B: the systolic blood pressure was measured in the total group, and subdivided in those with a normal eGFR and those with decreased eGFR. It was found that the systolic blood pressure increased at increasing tertiles of circulating dp-ucMGP both in the total group and in the group with low eGFR (p<0.001 in both cases), but not in those with normal eGFR.

FIG. 3C: similar observations as shown in FIG. 3B were made for diastolic blood pressure.

FIG. 3D: the number of subjects with hypertension was plotted in each of the dp-ucMGP tertiles for the total group, the normal eGFR and the low eGFR group. It is clear that hypertension was almost exclusively found in the low eGFR group, and that the number of hypertensive subjects increased at increasing dp-ucMGP.

### Example 4

All subjects in the FLEMENGHO cohort were classified according to their renal function. The largest groups were those with a normal (stage 1) or slightly decreased eGFR (stage 2). In FIG. 4 the association is shown between circulating matrix Gla protein levels and stage of chronic kidney disease. Box plots represent the median, interquartile range, and 5th to 95th percentile interval for dp-ucMGP in all participants (n=1159) and in participants with stages 1 (n=549), 2 (n=550), and 3 (n=60) according to the National Kidney Foundation (KDOQI) guideline (estimated glomerular filtration rates: 90, 60-89, 30-59 mL/min/1.73 m², respectively. The highest average dp-ucMGP level was found in the 60 subjects in the stage 3 group, and the difference with any of the other groups was statistically significant at p<0.001.

### CONCLUSIONS

1. We have demonstrated that vitamin K-insufficiency is a risk factor for impaired nutrient, waste and gas exchange. In this way it may be associated with the occurrence or progression of a wide variety of diseases.

2. High vitamin K intake may thus prevent or cure diseases of widely different nature, for instance chronic kidney disease, macula degeneration, left ventricular dysfunction, food malabsorption, pulmonary capillary dysfunction, male infertility, erectile dysfunction and vascular dementia.

3. Poor vitamin K status must be regarded as an independent risk factor for these diseases, the common denominator of which is poor capillary function or impaired capillary permeability.

4. We have demonstrated the beneficial effect of high vitamin K intake on the structure and integrity of the microvascular system as observed by non-mydriatic retinal photography, clearly resulting in improved microvascular health (less breaks or occlusions, less leakage) in the retina.

5. We have demonstrated a higher capillary density (number of vessels per mm³ of tissue) in subjects who are adequate in vitamin K compared to those who are insufficient. This was performed by sublingual sidestream dark field imaging. So the better nutrient supply is not only brought about by an enhanced blood flow, but also by more capillaries per unit of tissue.

6. With the same technique, we have demonstrated a thicker (=healthier) glycocalyx in the capillaries, which ensures a better exchange of nutrients from the blood stream into the extravascular compartment (i.e. the surrounding tissue). At present it is getting increasingly clear that the glycocalyx plays a dominant role in this process.

7. We have demonstrated that the unique capillary function, i.e. the exchange of nutrients, waste and gases between the blood stream and the extravascular compartment, is greatly enhanced by vitamin K. This was performed by measuring the renal estimated glomerular filtration rate (eGFR) which is the gold standard for capillary function.

8. Without wanting to be bound to any theory we believe a plausible mechanism for the surprising observations disclosed in the present invention may be explained by the presence of the vitamin K-dependent protein MGP in the walls of the microvasculature including the capillaries.

The present disclosure is to be considered as in all respects illustrative and not restrictive, the scope of the invention being indicated by the appended claims, and all changes which come within the meaning and range of equivalency are intended to be embraced therein.

1. A method of treatment for improving the exchange of nutrients, oxygen, carbon dioxide and waste between the blood stream and adjacent tissues across the capillary wall in the microvasculature of a mammalian subject, preferably a human subject, thus optimizing microvascular integrity and capillary structure, morphology, density and function, which comprises administering to said subject an effective amount of vitamin K.

2. A method according to claim 1, wherein said vitamin K is selected from phylloquinone (vitamin K1) and menaquinone (vitamin K2), preferably one of the menaquinones (vitamin K2) and combinations thereof, more preferably one of the long-chain menaquinones MK-7, MK-8, MK-9, MK-10, and combinations thereof.

3. A method according to claim 1, wherein said vitamin K is synthetically prepared.

4. A method according to claim 1, wherein said vitamin K is administered in addition to the normal dietary intake of vitamin K as a functional food, a food supplement, a probiotic or a pharmaceutical preparation.

5. A method according to claim 1, wherein said vitamin K is administered orally in a dose range between 5 and 5000 μg day, preferably between 25 and 1000 g/day, more preferably between 50 and 500 g/day and most preferably between 100 and 250 g/day.

6. A method according to claim 1, wherein said human subject suffers from a disease associated with impaired morphology or function of the microvasculature, in particular impaired capillary morphology or capillary dysfunction.

7. A method according to claim 6, wherein said microvasculature is restricted to the capillary bed.

8. A method according to claim 1, wherein said human subject suffers from a disease selected from renal disease, impaired vision or macula degeneration, left ventricular dysfunction, vascular dementia or cognitive decline, food malabsorption, COPD, pulmonary capillary dysfunction, impaired gas exchange efficiency, infertility or erectile dysfunction, diabetic necrosis and peripheral neuropathy.

9. A pharmaceutical or nutraceutical composition for use in the treatment of improving the exchange of nutrients, oxygen, carbon dioxide and waste between the blood stream and adjacent tissues across the capillary wall in the microvasculature of a mammalian subject, preferably a human subject, suffering from a disease selected from the group of diseases associated with impaired morphology or function of the microvasculature, in particular impaired capillary morphology or capillary dysfunction, renal disease, impaired vision or macula degeneration, left ventricular dysfunction, vascular dementia or cognitive decline, food malabsorption, COPD, pulmonary capillary dysfunction, impaired gas exchange efficiency, infertility or erectile dysfunction, diabetic necrosis and peripheral neuropathy, which comprises as active ingredient at least vitamin K.

10. A pharmaceutical or nutraceutical composition according to claim 9 wherein said vitamin K is used for administration:
a. in combination with at least one other food or food supplement known to improve cardiovascular health such as antioxidants, omega-3 fatty acids, niacin, coenzyme Q, inositol, stands, sterols, or L-arginine; or
b. in combination with at least one pharmaceutically or nutraceutically active product such as beta blockers, corticosteroids, ACE inhibitors or cholesterol lowering medication.

11. Use of the plethysmographic acceleration pulse wave technique as a screening method for evaluating the risk of vitamin K insufficiency or the risk of developing vitamin K-insufficiency related diseases such as vascular calcification, diabetes, metabolic syndrome, pulmonary disease, chronic kidney disease, macula degeneration, osteoporosis and osteoarthritis.
